(19) United States
(12) Patent Application Publication
(10) Pub. No.: US 2026/0263965 A1
(43) Pub. Date: Sep. 10, 2026

(54) PROCESS FOR AT LEAST PARTIALLY REMOVING ALDEHYDES FROM A COMPOSITION CONTAINING AT LEAST ONE COMPOUND HAVING AT LEAST ONE ALKYLENE OXIDE UNIT

(71) Applicant: ZSCHIMMER & SCHWARZ CHEMIE GMBH, Lahnstein/Rhein (DE)

(72) Inventor: ULLI STIER, Backnang (DE)

(21) Appl. No.: 19/489,297

(22) PCT Filed: Apr. 16, 2024
(86) PCT No.: PCT/EP2024/060252

§ 371 Date: Dec. 2, 2025

(30) Foreign Application Priority Data
Jun. 6, 2023 (EP) 23177465.4

(51) Int. Cl. B01D 15/36; B01D 15/38; C07C 41/58

(52) U.S. Cl. CPC B01D 15/362; B01D 15/3876; C07C 41/58

(57) ABSTRACT

The invention relates to a process for at least partially removing aldehydes from a composition, the process comprising the following steps:
a) contacting the composition with at least one cation exchanger and/or an acid at a temperature of 20.0° C. to 250.0° C.;
b) at least partially separating the aldehydes from the composition and, optionally, treatment of the separated aldehydes, selected from condensation, absorption, adsorption, chemical bonding, chemical reaction, oxidation and pyrolysis;
   whereby a composition having a diminished content of aldehydes is obtained;
   wherein the composition to be treated in step a) contains at least one compound having at least one alkylene oxide unit in its chemical structure and a molecular weight of 200 g/mol or more, and
   contains 3.5 wt % or less of alcohols having 1 to 6 carbon atoms, based on the total weight of the composition.

The invention also relates to a composition containing at least one compound having at least one alkylene oxide unit in its chemical structure and a molecular weight of 200 g/mol or more, the total aldehyde content being 1000 ppm or less.

The invention relates to a process for at least partially removing aldehydes from a composition resulting in chemical products having a low aldehyde content. Chemical products are brought into contact with at least one cation exchanger and/or an acid, and the aldehydes are subsequently removed from the product by means of separation processes. As a result, products can be prepared having an aldehyde content that has been diminished by about 10% of its initial value.

### BACKGROUND OF THE INVENTION

Aldehydes, like formaldehyde, acetaldehyde, propionaldehyde and butyraldehyde, are unwanted by-products when reacting, for example, alkenyl oxides (like ethylene oxide, propylene oxide, butylene oxide) to alkylene oxide adducts like polyalkylene glycols and non-ionogenic surfactants which can only be diminished to a certain extent by process parameters like temperature. Due to regulatory requirements or the usage of the aforementioned products in critical applications (e. g. sanitary products), a diminution of aldehydes can become necessary.

### BACKGROUND ART

EP 0 638 538 A1 describes the removal of formaldehyde from an aqueous acetic acid solution by adding methanesulfonic acid (and a polyol, if required). According to U.S. Pat. No. 5,440,058, formaldehyde is converted into the sodium salt of hydroxy methanesulfonic acid by adding NaHSO₃. EP 0 309 915 A1 describes the removal of formaldehyde from an aqueous butynediol solution by adding an “acidic-acting agent” like methanesulfonic acid or a cation exchanger, and methanol, producing dimethylformal which is distilled off.

According to U.S. Pat. No. 6,187,973, an aqueous ethylene glycol solution containing aldehydes like formaldehyde, acetaldehyde and the same, is brought into contact with a solid bisulfite-treated highly basic anion exchanger resin, and a solution with a diminished aldehyde content is separated.

CA 1330350 discloses a process for the purification of ethylene glycol using a basic ion exchanger resin containing bisulfite or hydroxyl groups. The anion exchanger resin adsorbs aldehyde impurities from the ethylene glycol.

DE 1668052 describes a method for purifying glycols to remove discoloration causing substances by way of cation exchangers, wherein, in the purification method, glycols are used that are basically free of formaldehyde.

The removing of aldehydes in the aforementioned patent documents is limited to low-molecular chemical compounds.

WO 2019/097407 discloses polymeric resins functionalized with primary amines for removing aldehydes. The resins are capable of removing aliphatic and aromatic aldehydes from a plurality of feeding streams. The resins form covalent imine bonds with aldehyde impurities. By exclusively using a basic ion exchanger, as claimed in WO 2019/0974707, no significant diminution of aldehydes is achieved with alkoxylates, however.

EP 3 228 649 A1 describes a method for processing alkali-catalyzed alkoxylation products by using sulfonic acid ion exchangers comprising the provision of a mixture containing the alkali-catalyzed alkoxylation product to be processed, alcohols having 1 to 4 carbon atoms, and water, treating this mixture with a sulfonic acid cation exchanger at >40° C., as well as separating the alkoxylation product from the mixture treated in this way.

### TECHNICAL OBJECTIVE

Thus, the technical objective of the present invention was to provide a process by which a significant diminution of aldehydes in alkoxylates can be achieved, particularly of alkoxylates having a higher molecular weight. Moreover, it was a preferred objective to remove, beside aldehydes, further unwanted impurities from alkoxylates. Particularly, it was an objective of the present invention to diminish or avoid, respectively, the use of solvents like alcohols in processes for purifying alkoxylates or in processes for removing aldehydes from alkoxylates, respectively.

### DESCRIPTION OF THE INVENTION

The present invention provides a process for at least partially removing aldehydes from a composition, the process comprising the following steps:
a) contacting the composition with at least one cation exchanger and/or an acid at a temperature of 20.0° C. to 250.0° C.;
b) at least partially separating the aldehydes from the composition and, optionally, treatment of the separated aldehydes, selected from the group consisting of condensation, absorption, adsorption, chemical bonding, chemical reaction, oxidation and pyrolysis;
whereby a composition having a diminished content of aldehydes is obtained;
wherein the composition to be treated in step a) contains at least one compound having at least one alkylene oxide unit in its chemical structure and a molecular weight of 200 g/mol or more, and
3.5 wt % or less of alcohols having 1 to 6 carbon atoms, based on the total weight of the composition.

In a preferred process, the composition to be treated in step a) (which contains at least one compound having at least one alkylene oxide unit in its chemical structure and a molecular weight of 200 g/mol or more) contains 3.0 wt % or less, further preferably 2.5 wt % or less, even further preferably 2.0 wt % or less, especially preferably 1.5 wt % or less, particularly preferably 1.0 wt %, very particularly preferably 0.5 wt % or less, most preferably 0.1 wt % or less of alcohols having 1 to 6 carbon atoms, based on the total weight of the composition. It is particularly preferred that the composition to be treated in step a) does not contain any (i. e. 0.0 wt %) of alcohols having 1 to 6 carbon atoms.

In another preferred process, the composition (which contains at least one compound having at least one alkylene oxide unit in its chemical structure and a molecular weight of 200 g/mol or more) to be treated in step a) contains 3.5 wt % or less, preferably 3.0 wt % or less, further preferably 2.5 wt % or less, even more preferably 2.0 wt % or less, especially preferably 1.5 wt % or less, particularly preferably 1.0 wt % or less of a solvent having 1 to 6 carbon atoms based on the total weight of the composition. Here, an alcohol having 1 to 6 carbon atoms represents a solvent having 1 to 6 carbon atoms.

In an alternative process, the composition (which contains at least one compound having at least one alkylene oxide unit in its chemical structure and a molecular weight of 200 g/mol or more) to be treated in step a) contains 3.5 wt % or less, preferably 3.0 wt % or less, further preferably 2.5 wt % or less, even further preferably 2.5 wt % or less, further preferably 2.0 wt % or less, even further preferably 2.0 wt % or less, even further preferably 1.5 wt % or less, especially preferably 1.5 wt % or less, and particularly preferably 1.0 wt % or less of a solvent selected from the group consisting of aliphatic ethers, cyclic ethers, hydrocarbons, ketones, alcohols having 1 to 6 carbon atoms, based on the total weight of the composition.

In a preferred process, the aldehydes are selected from the group of aldehydes having a molecular weight of 200 g/mol or less. Further preferably, the aldehydes are selected from the group consisting of formaldehyde, acetaldehyde, propionaldehyde and butyraldehyde. This applies to the aldehydes to be removed before conducting the process, to the aldehydes separated by said process as well as to the diminished content of aldehydes, if present.

Therefore, the present invention provides a process which does not only allow for removing aldehydes from chemical products and compositions, respectively, but also from those containing glycols and (poly)alkylene oxide compounds with higher molecular weights. Furthermore, within the scope of the invention, the inventor discovered that other impurities such as dioxane and metal ions can be removed from such compositions. Moreover, the inventor of the present invention discovered that, contrary to methods of the prior art, solvents like alcohols, in particular alcohols having 1 to 6 carbon atoms, do not have to be added to the composition to be treated in order to achieve the desired purification. The process according to the present invention is advantageous because the respective alcohols do not have to be provided and neither be separated and recycled. Thereby, effort and cost for the process are diminished. The same preferably applies to the aforementioned solvents as well.

The term composition to be purified or purified composition having a diminished content of aldehydes, respectively, wherein the composition to be purified or the purified composition, respectively, each contains at least one compound having at least one alkylene oxide unit in its chemical structure and a molecular weight of 200 g/mol or more, has to be understood as a liquid composition or a liquid, respectively, preferably a solution or an emulsion. Here, the liquid aggregate state of the composition is at least existing under the conditions of the process according to the invention, in particular at the temperature that is applied during the process.

Surprisingly, it was found that by treating compositions or chemical products, respectively, with at least one cation exchanger and/or an acid at temperatures of 20° C. to 250° C., preferably at temperatures of 40° C. to 140° C. (step a) and a subsequent separation process for removing aldehydes (step b), products are obtained having an aldehyde content that is diminished by at least 10% of the initial value. In a particularly preferred process, step a) is carried out at a temperature of 40° C. to 140° C., further preferably at a temperature of 60° C. to 120° C., even further preferably at a temperature of 80° C. to 120° C., and particularly preferably at a temperature of 85° C. to 120° C.

Said process according to the invention can be operated continuously or discontinuously.

In another preferred process, the cation exchanger used in step a) is an acidic cation exchanger that contains acid groups selected from the group consisting of —SO₃H, —COOH and —OP(OH)₃. Therefore, the cation exchangers preferably have sulfonic acid groups (—SO₃H), carboxy groups (—COOH) or orthophosphoric acid groups (—OP(OH)₃) as functional groups of the unloaded form.

The matrix of the ion exchanger resins can, for example, be obtained by condensation (phenol-formaldehyde matrix) or by polymerization (polystyrene matrix, a matrix of copolymers of styrene with divinylbenzene, a polyacryl matrix, a matrix of copolymers of acrylates, methacrylates or acrylonitrile with divinylbenzene). The ion exchangers employed according to the invention may be present in different forms such as solid grains and particles (pellets, beads), furthermore as membranes, films, fibers and tissues. Preferably, as forms of the ion exchanger resins, solid grains and particles (pellets, beads) are used.

In a continuous operation, the ion exchangers may, for example, be present in columns or containers. The columns or containers may have nozzle floors or drainage systems for the entry and/or exit of the treated chemical product. In operation, the ion exchanger may be operated continuously in different implementations, for example in counter-current, co-current, layered-bed, multi-chamber, double-flow, sandwich or mixed-bed ion exchanger.

In a discontinuous operation, the ion exchangers may, for example, be present in their free form within the composition, i. e. the chemical product, or in perforated containers that get into contact with the composition.

In another preferred process, the acid used in step a) is selected from the group consisting of sulfuric acid, phosphoric acid, phosphonic acid, phosphinic acid, hydrochloric acid, methanesulfonic acid, toluenesulfonic acid and alkylbenzene sulfonic acid, preferably methanesulfonic acid. The concentration of the employed acids is preferably 10 wt % or less, further preferably 5.0 wt % or less, especially preferably 2.0 wt % or less, and most preferably 1.0 wt % or less. In the process that uses an acid in step a), it is particularly preferred that the compound having at least one alkylene oxide unit in its chemical structure and a molecular weight of 200 g/mol or more is a compound with a carbon backbone.

As stated above, according to the invention, in step b) a partial separation of aldehydes from the composition is carried out, preferably followed by a treatment of the separated aldehydes, selected from the group consisting of condensation, absorption, adsorption, chemical bonding, chemical reaction, oxidation and pyrolysis. The preferred step of treating the separated aldehydes carried out after step b) is selected from the group consisting of condensation, absorption, adsorption, chemical bonding, chemical reaction, oxidation and pyrolysis and can be referred to as step c).

In another preferred embodiment, step b) is carried out by way of a thermal separation process selected from the group consisting of distillation process, rectification process, stripping process and flash evaporation process. The aforementioned thermal separation processes may also be used in combination. Furthermore, solvents like water, or entrainers may be added to the composition to be purified in order to enhance the separation efficiency.

In process step b), i. e. the thermal separation process selected from the group consisting of distillation process, rectification process, stripping process and flash evaporation process, the aldehydes are removed from the composition (i. e. from the product (stream)) via the gas phase. In a particularly preferred embodiment, the distillation, rectification or stripping process is carried out under vacuum. In the stripping process, as stripping gas may be used, for example, steam, nitrogen, carbon dioxide, air or argon. The substances contained in the gas stream, such as aldehydes, can at least partially be removed from the gas phase by way of common methods of waste gas purification or waste air purification. It is understood that biological waste gas purification is also included. The substances contained in the gas phase, such as aldehydes, may, for example, be condensed and thereby collected, or, for example, be absorbed by an absorption medium such as a washing solution. In case the absorption capability of the absorption medium such as water is not sufficient, the absorption can be complemented by chemical reaction using chemical adjuvants (chemisorption). Furthermore, the substances contained in the gas stream may be adsorbed by an adsorption medium such as activated carbon, or, for example, chemically bound by an ion exchanger or chemically converted. In general, chemical conversion includes catalytic or non-catalytic processes. In a non-catalytic chemical process, aldehyde adducts can be formed using chemical adjuvants such as bisulfite in order to at least partially remove the aldehydes. Furthermore, the substances in the gas phase can be decomposed by oxidation or pyrolysis, including the process of reburning. It is understood that the methods mentioned above: condensation, absorption, adsorption, chemical bonding, chemical conversion, oxidation, pyrolysis may be used in combination.

Exemplary thermal separation devices that may be operated under vacuum, are distillation columns, rectification columns, evaporators such as forced circulation and natural circulation evaporators, horizontal evaporators, rotary evaporators, falling-film evaporators, thin-film evaporators, short-path evaporators or degassers or spray dryers, wherein preferably a waste gas purification unit or a waste air purification unit is installed downstream of the evaporation unit. As absorbers may be employed, among others, Venturi scrubbers, spray washers, jet scrubbers or swirl washers. As adsorbers may be employed, among others, fixed-bed, rotary-bed, fluidized-bed or moving-bed adsorbers. In order to separate gaseous substances like aldehydes as liquids, cooling units for condensation may be installed downstream of the thermal separation devices. Furthermore, afterburners may be installed downstream of the thermal separation devices. In the case of biological waste gas purification, bio-scrubbers, bio filters, bio-trickling filter-bed reactors may be used, among others.

In an alternative preferred embodiment of the process, step b) is carried out by contacting an ion exchanger having amino- or bisulfite-functional groups, wherein step b) is preferably carried out at a temperature of 20° C. to 250° C. Here, the aldehydes are bound to the ion exchanger and removed from the composition (i. e. from the product (stream)).

Amino-functional ion exchangers may, for example, be present as a matrix based on a branched polystyrene that has functional amino groups. The amino-functional ion exchangers are brought into contact with the chemical product (-stream) in order to bind the aldehydes contained therein to the amino groups and to remove them from the product (-stream) in this way. Primary amino groups of the resins, for example, can covalently bind the aldehydes by forming an imine compound.

Bisulfite-functional ion exchangers may, for example, be prepared by treating a highly basic ion exchanger resin having hydroxy groups with a bisulfite solution. By this treatment, the basic ion exchanger is converted into the bisulfite form with (resin-HSO₃⁻) groups. The preparation of the bisulfite form is illustrated as follows in an exemplary way by means of treating a highly basic ion exchanger resin with a sodium bisulfite solution:

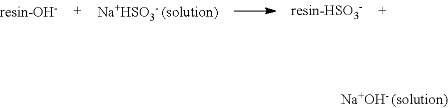

When getting into contact with the chemical product, the (resin-HSO₃⁻) groups can bind the aldehydes contained therein and thus remove them from the product (-stream), wherein the following reaction mechanism has to be expected, using the elimination of formaldehyde as an example:

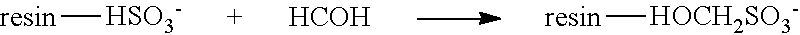

In another preferred process, the content of compounds having at least one alkylene oxide unit in their chemical structure and a molecular weight of 200 g/mol or more in the employed composition is at least 10 wt %, based on the total weight of the composition, preferably at least 20 wt %, preferably at least 50 wt %, further preferably at least 80%, even further preferably at least 90.0 wt %, even further preferably at least 95.0%, even further preferably at least 98.0 wt %, even further preferably at least 99.0 wt %, and most preferably at least 99.5 wt %. These contents preferably also apply to the composition obtained after step b) that has a diminished aldehyde content.

The process according to the invention allows for the removal of aldehydes not only in aqueous but also from concentrated compositions or chemical products, respectively. Thus, the water content of the employed composition is preferably 90 wt % or less, based on the total weight of the composition, further preferably 70 wt % or less, even further preferably 50 wt % or less, further preferably 40 wt % or less, further preferably 30 wt % or less, further preferably 20 wt % or less, even further preferably 10.0 wt % or less, even further preferably 5.0 wt % or less, even further preferably 2.0 wt % or less, even further preferably 1.0 wt % or less, and most preferably 0.5 wt % or less. These water contents preferably also apply to the composition obtained after step b) that has a diminished aldehyde content.

In another preferred embodiment of the invention, the aldehydes to be removed from the compositions have at least one aldehyde group in their structure. The aldehydes may have further functional groups and may also contain heteroatoms in their structure. In a preferred process, the aldehydes are selected from the group consisting of aldehydes having a molecular weight of 200 g/mol or less. Further preferably, the aldehydes are selected from the group consisting of formaldehyde, acetaldehyde, propionaldehyde and butyraldehyde.

By using the process according to the invention, an outstanding purification is achieved, particularly with regard to aldehydes. In a preferred process, after step b), a composition is obtained the total aldehyde content of which is diminished by at least 10% of the initial value, further preferably by at least 30%, preferably by at least 50%, further preferably by at least 80%, even further preferably by at least 90%, even further preferably by at least 95%, even further preferably by at least 98%, and even further preferably by at least 99%. In a preferred process, the total aldehyde content is defined as the total content of aldehydes that have a molecular weight of 200 g/mol or less. In another preferred process, the total aldehyde content is defined as the total content of aldehydes that are selected from the group consisting of formaldehyde, acetaldehyde, propionaldehyde and butyraldehyde.

In a particularly preferred process, after step b) a composition is obtained the total aldehyde content of which is diminished to 1000 ppm or less, further preferably to 900 ppm or less, preferably 800 ppm or less, further preferably to 500 ppm or less, even further preferably to 400 ppm or less, even further preferably to 300 ppm or less, even further to 200 ppm or less, even further preferably to 150 ppm or less, even further preferably to 100 ppm or less, even further preferably to 50 ppm or less, and most preferably diminished to 10 ppm or less. In a particularly preferred embodiment, the residual aldehyde contents mentioned herein refer to a composition whose content of compounds having at least one alkylene oxide unit in their chemical structure and a molecular weight of 200 g/mol or more, is at least 10 wt %, based on the total weight of the composition, particularly preferred at least 50 wt %. In a preferred process, the total aldehyde content is defined as the total content of aldehydes that have a molecular weight of 200 g/mol or less. In another preferred process, the total aldehyde content is defined as the total content of aldehydes selected from the group consisting of formaldehyde, acetaldehyde, propionaldehyde and butyraldehyde. The designation “ppm” refers to the total aldehyde content in mg per 1 kg of the composition.

In another most preferred process, after step b) a composition is obtained whose content of compounds that have at least one alkylene oxide in their chemical structure and a molecular weight of 200 g/mol or more, is at least 10 wt %, based on the total weight of the composition, particularly preferably at least 50%, and whose total aldehyde content is diminished to 1000 ppm or less, further preferably to 900 ppm or less, preferably to 800 ppm or less, further preferably to 500 ppm or less, even further preferably to 400 ppm or less, even further preferably to 300 ppm or less, even further preferably to 200 ppm or less, even further preferably to 150 ppm or less, even further preferably to 100 ppm or less, even further preferably to 50 ppm or less, and most preferably diminished to 10 ppm or less, wherein the total aldehyde content is defined as the total content of aldehydes that have a molecular weight of 200 g/mol or less, wherein the total aldehyde content is further preferably defined as the total content of aldehydes that are selected from the group consisting of formaldehyde, acetaldehyde, propionaldehyde and butyraldehyde. The designation “ppm” refers to the total content of aldehydes in mg per 1 kg of the composition.

Beside the separation of aldehydes, it was surprisingly found that dioxane can also be removed using the process according to the invention. Regarding dioxane, the following description primarily refers to 1,4-dioxane. There are, however, other dioxane isomers that are under consideration as well. Therefore, the dioxane comprises one or more substances selected from the group consisting of 1,2-dioxane, 1,3-dioxane and 1,4-dioxane, preferably 1,4-dioxane. Thus, in another preferred process, step b) is carried out by a thermal separation process selected from the group consisting of distillation process, rectification process, stripping process and flash evaporation process, wherein after step b) a composition is obtained the dioxane content of which is diminished to 1000 ppm or less, further preferably to 900 ppm or less, preferably to 800 ppm or less, further preferably to 500 ppm or less, even further preferably to 400 ppm or less, even further preferably to 300 ppm or less, even further preferably to 200 ppm or less, even further preferably to 150 ppm or less, even further preferably to 100 ppm or less, even further preferably to 50 ppm or less, and most preferably diminished to 10 ppm or less. In a particularly preferred embodiment, the residual contents of dioxane mentioned herein refer to a composition whose content of compounds containing at least one alkylene oxide unit in their chemical structure and having a molecular weight of 200 g/mol or more, is at least 10 wt %, based on the total weight of the composition, particularly preferably at least 50 wt %. The designation “ppm” refers to the amount of dioxane in mg per 1 kg of the composition.

Beside the separation of aldehydes, the alkoxylation catalyst, such as NaOH or KOH, or metal-cyanide-complex catalysts as shown in U.S. Pat. No. 5,158,922 or US 2003/0119663, can also be removed by the process of the invention by using a cation exchanger in step a). Therefore, in another preferred process, in step a) the composition is brought into contact with at least one cation exchanger, whereby after step b) a composition is obtained the metal ion content of which is diminished to 1000 ppm or less, further preferably to 900 ppm or less, preferably to 800 ppm or less, further preferably to 500 ppm or less, even further preferably to 400 ppm or less, even further preferably to 300 ppm or less, even further preferably to 200 ppm or less, even further preferably to 150 ppm or less, even further preferably to 100 ppm or less, even further preferably to 50 ppm or less, and most preferably diminished to 10 ppm or less. In a particularly preferred embodiment, the residual contents of metal ions mentioned herein refer to a composition whose content of compounds having at least one alkylene oxide unit in their chemical structure and a molecular weight of 200 g/mol or more, is at least 10 wt %, based on the total weight of the composition, particularly preferably at least 50 wt %. In a preferred embodiment, the metal ions are selected from the group consisting of Na(I), K(I), Zn(II), Fe(II), Fe(III), Co(II), Co(III), Ni(II), Mn(II), Mn(III), Ir(III), Rh(III), Ru(II), V(IV), V(V), Co(II), Sn(II), Pb(II), Mo(IV), Mo(VI), Al(III), V(IV), V(V), Sr(II), W(IV), W(VI), Cu(II), Cr(II) and Cr(III). The designation “ppm” refers to the amount of metal ions in mg per 1 kg of the compound.

Compounds Having at Least One Alkylene Oxide Unit in their Chemical Structure and a Molecular Weight of 200 g/Mol or More

In another particularly preferred process, the composition or the compounds having at least one alkylene oxide unit in their chemical structure and a molecular weight of 200 g/mol or more, are obtained from polymerizing alkylene oxides or from an alkoxylation process. In the present application, the specification of the molecular weight of “compounds having at least one alkylene oxide unit in their chemical structure and a molecular weight of 200 g/mol or more”, always refers to the number-average molecular weight. The terms “number-average molecular weight” and “average molecular weight” are used synonymously herein. Furthermore, the terms “molecular weight” and “molar mass” are used synonymously.

The number-average molecular weight can, for example, be calculated from the amounts that are employed in synthesis. If, for example, a fatty alcohol having 5 ethylene oxide (EO) units is to be synthesized, for the synthesis of this alkoxylate an appropriate mass ratio of fatty alcohol to ethylene oxide (EO) is weighed in, one EO unit being equivalent to 44.05 g/mol. The number-average molecular weight of the ethoxylated fatty alcohol is calculated by adding the molecular weight of the fatty alcohol (e. g. 1-octadecanol: 270.5 g/mol) and the 5 ethylene oxide units (5×44.05 g/mol).

The number-average molecular weights can be determined by cryoscopy, ebullioscopy, vapor pressure osmometry (for molar masses up to about 50,000 g/mol), osmometry (for molar masses up to about 10,000 g/mol) or NMR spectroscopy. Furthermore, from the molar mass distribution, the number-average molecular weight can be determined by way of gel permeation chromatography (GPC) and mass spectrometry (MALDI-TOF).

Furthermore, the average molecular weight can be determined according to the OECD guidelines (Guidelines for the Testing of Chemicals, section 1) according to Test no. 118 (Determination of the Number-Average Molecular Weight and the Molecular Weight Distribution of Polymers using Gel Permeation Chromatography).

As alkylene oxides, compounds may be used that have the following formula (I):

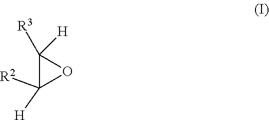

Wherein R² and R³ independently of each other represent hydrogen or a hydrocarbon residue; wherein the hydrocarbon residue may be a cycloaliphatic hydrocarbon residue, preferably a linear or branched hydrocarbon residue, particularly a hydrocarbon residue having 1 to 20, preferably 1 to 6 carbon atoms, particularly preferably a methyl, ethyl or phenyl residue. The residues R² and R³ may also be part of a cyclic group, R² and R³ then form a bivalent residue. The hydrocarbon residues R² and R³ may in turn bear functional groups such as halogens, hydroxy groups and glycidyloxypropyl groups. Included in such alkylene oxides are epichlorohydrin, 2,3-epoxy-1-propanol as well as polyfunctional epoxy compounds like 1,2-ethyl-, 1,4-butyl- and 1,6-hexyl glycidyl ether. Preferably, at least one of the two residues R² or R³ is hydrogen. It is particularly preferred that the employed alkylene oxides are selected from the group consisting of ethylene oxide, propylene oxide, 1,2- or 2,3-butylene oxide, isobutylene oxide, 1,2-dodecen oxide, cyclohexene oxide, vinylcyclohexene oxide and styrene oxide.

As alkylene oxides, glycidyl compounds such as glycidyl ethers or glycidyl esters may also be used, the at least one glycidyloxypropyl group of which is bound via an ether or ester function to a linear or branched alkyl residue having 1 to 24 carbon atoms, an aromatic or cycloaliphatic residue. This class of compounds includes, for example, allyl-, butyl-, 2-ethylhexyl-, cyclohexyl-, benzyl-, C₁₂/C₁₄ fatty alcohol-, phenyl-, p-tert-butylphenyl- and o-cresyl glycidyl ethers. The glycidyl esters that are preferably used include, for example, glycidyl methacrylate, glycidyl acrylate and neodecanoic acid glycidyl ester.

In another particularly preferred process, the compounds having at least one alkylene oxide unit in their chemical structure and a molecular weight of 200 g/mol or more, have at least one (poly)alkylene oxide group with at least 1, preferably with at least 2, preferably with at least 3, further preferably with at least 4 and even further preferably with at least 5 alkylene oxide units, even further preferably with at least 6, even further preferably with at least 8, even further preferably with at least 10, even further preferably with at least 12, even further preferably with at least 15, even further preferably with at least 50, and most preferably with at least 100 alkylene oxide units. The upper limit of alkylene oxide units is not particularly limited. In preferred embodiments, the number of alkylene oxide units is 500 or fewer, further preferably 200 or fewer, and even further preferably 150 or fewer. Said alkylene oxide units are preferably selected from the group consisting of ethylene oxide, propylene oxide and butylene oxide, particularly preferably ethylene oxide and/or propylene oxide.

In another particularly preferred process, the compounds having at least one alkylene oxide in their chemical structure and a molecular weight of 200 g/mol or more, have at least one (poly)alkylene oxide group of the formula —(C₂H₄—O)ₗ—(C₃H₆—O)ₘ—(C₄H₈—O)ₙ— or —(C₂H₄—O)ₗ—(C₃H₆—O)ₘ—(C₄H₈—O)ₙ—H, wherein l, m and n independently of each other are 0 or more, and wherein the sum of l, m and n is 1 or more, preferably, the sum of l, m and n is 2 or more, preferably, the sum of l, m and n is 3 or more, further preferably 4 or more, even further preferably 5 or more, even further preferably 6 or more, even further preferably 8 or more, even further preferably 10 or more, even further preferably 12 or more, particularly preferably 15 or more, especially preferably 50 or more, and most preferably 100 or more. The upper limit of the alkylene oxide units is not particularly limited. In further embodiments, it is preferred that the sum of l, m and n is a number from 1 to 500, it is further preferred that the sum of l, m and n is a number from 1 to 200, and it is even further preferred that the sum of l, m and n is a number from 1 to 150. In another particularly preferred embodiment, the sum of l, m and n is a number from 2 to 500, preferably, the sum of l, m and n is a number from 2 to 200, and it is particularly preferred that the sum of l, m and n is a number from 2 to 150.

In another particularly preferred embodiment, the compounds having at least one alkylene oxide unit in their chemical structure and a molecular weight of 200 g/mol or more, have at least one polyoxyalkylene group of the formula —(CₓH₂ₓ—O)ₓ— or —(CₓH₂ₓ—O)_{y}—H, wherein x is 2, 3 or 4 and y is 1 or more, preferably, y is 2 or more, 3 or more, 4 or more, 5 or more, 6 or more, 8 or more, 10 or more, 12 or more, 15 or more, 50 or more, 100 or more. The upper limit of the alkylene oxide units is not particularly limited. In further embodiments, it is preferred that y is a number from 1 to 500, further preferably y is a number from 1 to 200, and even further preferably, y is a number from 1 to 150. In another particularly preferred embodiment, y is a number from 2 to 500, preferably, y is a number from 2 to 200, and it is particularly preferred that y is a number from 2 to 150.

In preferred embodiments, the molecular weight of the compounds having at least one alkylene oxide in their chemical structure, is 250 g/mol or more, preferably 300 g/mol or more, further preferably 400 g/mol or more, even further preferably 500 g/mol, especially preferably 1,000 g/mol or more, and particularly preferably 2,000 g/mol. The upper limit of the molecular weight of the compounds having at least one alkylene oxide unit in their chemical structure is not particularly limited. In preferred embodiments, the molecular weight of the compounds having at least one alkylene oxide unit in their chemical structure, is 100,000 g/mol or less, preferably 50,000 g/mol or less, further preferably 25,000 g/mol or less, even further preferably 20,000 g/mol or less, especially preferably 15,000 g/mol or less, and particularly preferably 12,000 g/mol or less.

In another particularly preferred process, the alkylene oxide units of the compounds having at least one alkylene oxide unit in their chemical structure and a molecular weight of 200 g/mol or more, are selected from the group consisting of ethylene oxide, propylene oxide and butylene oxide.

In another preferred embodiment of the process, the compounds having at least one alkylene oxide unit in their chemical structure and a molecular weight of 200 g/mol or more, represent (poly)alkylene oxide adducts with at least one further residue selected from the group consisting of carboxylate residues, carboxylic acid ester residues, carboxamide residues, phenol residues and alcohol residues. The alkylene oxide units are preferably selected from the group consisting of ethylene oxide, propylene oxide and butylene oxide, particularly preferably ethylene oxide and/or propylene oxide.

(Poly)alkylene oxide adducts in the sense of this description are reaction products of starting materials that can be alkoxylated, such as carboxylic acids like ethylhexanoic acid, benzoic acid, or fatty acids like caprylic acid, capric acid, lauric acid, cocoyl acid, palmitic acid, stearic acid, oleic acid, ricinoleic acid, or those having multiple carboxylic groups like citric acid, agaric acid and propane-1,2,3-tricarboxylic acid, trimellitic acid, trimesic acid, pyromellitic acid and mellitic acid, and mixtures thereof, carboxylic acid esters such as triglycerides like castor oil, rapeseed oil, soy oil, sunflower oil, coconut oil, or diglycerides like glyceryl dioleate, or monoglycerides like glyceryl monooleate, or sorbitan esters like sorbitan monolaurate, sorbitan monooleate, sorbitan trioleate, sorbitan monostearate, sorbitan sesquiisostearate, or polyglycerol fatty acid esters like polyglycerol cocoate, polyglycerol ricinoleat, polyglycerol oleate, or fatty acid esters like methyl oleate, carboxamides such as fatty acid amides like cocoyl acid amide, cocoyl acid monoethanol amide, cocoyl acid diethanol amide, oleic acid amide, phenols like alkyl phenols, tristyryl phenol, monovalent alcohols like methanol, ethanol, propanol, butanol, ethylhexanol, methoxyethanol, methyl diglycol, or fatty alcohols like lauryl alcohol, stearyl alcohol, oleyl alcohol or oxo alcohols like isotridecyl alcohol, linear or branched oxo alcohol-C₁₂-C₁₅, or cyclic alcohols such as cyclohexanol or Guerbet alcohols like butyl octanol, hexyl decanol and octyl decanol, or polyvalent alcohols like ethylene glycol, propylene glycol, butane diol, diethylene glycol, glycerol, polyglycerol, alkyl glycosides, sorbitol, mannitol, sorbitan, isosorbit, trimethylol propane, pentaerythritol, dipentaerythritol, and mixtures thereof. The (poly)alkylen oxide adducts have at least one alkylene oxide unit, i. e. they have 1-500, preferably 1-200, further preferably 1-150 alkylene oxide units. Said alkylene oxide units are preferably selected from the group consisting of ethylene oxide, propylene oxide and butylene oxide, particularly preferably ethylene oxide and/or propylene oxide. The described (poly)alkylene oxide adducts may be built from the same or different alkylene oxides, for example from block-like or statistically arranged ethylene oxide and propylene oxide, so that the present application also comprises such “mixed” alkylene oxide adducts.

In an alternative preferred embodiment of the process, the compounds having at least one alkylene oxide unit in their chemical structure and a molecular weight of 200 g/mol or more, are selected from the group consisting of polyglycols, polyalkylene glycols, block copolymers, carboxylic acid alkoxylates, carboxylic acid polyglycol esters, alkoxylated carboxylic acid esters, alkoxylated carboxamides, alkoxylated phenols and alcohol alkoxylates. The alkylene oxide units are preferably selected from the group consisting of ethylene oxide, propylene oxide and butylene oxide, particularly preferably ethylene oxide and/or propylene oxide.

Compounds having at least one alkylene oxide unit in their chemical structure and a molecular weight of 200 g/mol or more, are preferably derived from the group of the C₂-C₄ alkylene oxides, preferably C₂-C₃ alkylene oxides. Particularly preferably, the alkylene oxide units are derived from ethylene oxide or propylene oxide or a mixture thereof. In a particular embodiment, the compounds having at least one alkylene oxide unit in their chemical structure and a molecular weight of 200 g/mol or more, are polyglycols having the formula H—[O—C₂H₄]ₚ—[O—C₃H₆]_{q}—[O—C₄H₈]ᵣ—OH, wherein p, q and r independently of each other each are a number of 0 or higher, and the sum of p, q and r is a number of 5 or higher, preferably, the sum of p, q and r is a number of 6 or higher, preferably 8 or higher, further preferably 10 or higher, even further preferably 12 or higher, especially preferably 15 or higher, particularly preferably 50 or higher, and most preferably 100 or higher. The upper limit of the alkylene oxide units is not particularly limited. In further embodiments, it is preferred that the sum of p, q and r is a number from 5 to 500, it is further preferred that the sum of p, q and r is a number from 5 to 200, and even further preferably, the sum of p, q and r is a number from 5 to 150. In another particularly preferred embodiment, the sum of p, q and r is a number from 10 to 500, preferably, the sum of p, q and r is a number from 10 to 200, and particularly preferably, the sum of p, q and r is a number from 10 to 150.

In a particular embodiment, the compounds having at least one alkylene oxide unit in their chemical structure and a molecular weight of 200 mg/mol or more, are non-ionogenic surfactants. Exemplary non-ionogenic surfactants are selected from the group consisting of carboxylic acid alkoxylates, carboxylic acid glycol esters, alkoxylated carboxylic acid esters, alkoxylated carboxamides, alkoxylated phenols, block copolymers based on poly(alkylene oxides) and alcohol alkoxylates. It has to be understood that the aforementioned non-ionogenic surfactant classes may have further functional groups or heteroatoms in their structure.

Exemplary carboxylic acid alkoxylates and carboxylic acid polyglycol esters are based on 2-ethyl hexanoic acid, benzoic acid or fatty acid alkoxylates and fatty acid polyglycol esters, respectively, based on caprylic acid, capric acid, lauric acid, cocoyl acid, palmitic acid, stearic acid, oleic acid, ricinoleic acid, and mixtures thereof. Carboxylic acid alkoxylates and carboxylic acid polyglycol esters that may have multiple ester groups in their structure are, for example, alkoxylates or polyglycol esters based on citric acid, agaric acid and propane-1,2,3-tricarboxylic acid, trimellitic acid, trimesic acid, pyromellitic acid and mellitic acid, and mixtures thereof.

Exemplary alkoxylated carboxylic acid esters are alkoxylates based on triglycerides such as castor oil, rapeseed oil, soy bean oil, sunflower oil, coconut oil, or diglycerides such as glycerol dioleate, or monoglycerides such as glycerol monooleate, or sorbitan esters such as sorbitan monolaurate, sorbitan monooleate, sorbitan trioleate, sorbitan monostearate, sorbitan sesquiisostearate, or polyglycerol fatty acid esters such as polyglycerol cocate, polyglycerol ricinoleate, polyglycerol oleate, or fatty acid esters like methyl oleate, and mixtures thereof. Normally, the carboxylic acid esters used as starting materials have at least one hydroxy and/or carboxylate group or are partially saponified prior to alkoxylation. Furthermore, reacting the carboxylic acid esters to yield the respective alkoxylates is possible by way of an insertion alkoxylation process.

Exemplary alkoxylated carboxamides are alkoxylates based on fatty acid amides such as cocoyl acid amide, cocoyl acid monoethanolamide, cocoyl acid diethanolamide, oleamide, and mixtures thereof.

Exemplary alkoxylated phenols are alkoxylates based on alkylphenol derivatives such as n-propylphenols, isopropylphenols, butylphenols, amylphenols, hexylphenols, heptylphenols, octylphenols, nonylphenols, dodecylphenols, methylphenols (cresols), dimethylphenols (xylenols) and ethylphenols or tristyrylphenol, and mixtures thereof.

Exemplary block copolymers based on poly(alkylen) oxides are ethylene oxide/propylene oxide block polymers.

Exemplary alcohol alkoxylates are alkoxylated monovalent alcohols based on methanol, ethanol, propanol, butanol, ethyl hexanol, methoxy ethanol, methyl diglycol or fatty alcohol alkoxylates based on lauryl alcohol, stearyl alcohol, oleyl alcohol, or oxo alcohol alkoxylates based on isotridecyl alcohol, linear or branched oxo alcohol-C₁₂-C₁₅, or alkoxylated cyclic alcohols such as cyclohexanol or Guerbet alcohol alkoxylates based on butyl octanol, hexyl decanol and octyl decanol, or alkoxylated polyvalent alcohols, polyols, based on ethylene glycol, propylene glycol, butanediol, diethylene glycol, glycerol, polyglycerol, alkyl glycosides, sorbitol, mannitol, sorbitan, isosorbit, trimethylol propane, pentaerythritol, dipentaerythritol, and mixtures thereof.

The present invention furthermore provides a composition containing at least one compound having at least one alkylene oxide unit in its chemical structure and a molecular weight of 200 g/mol or more, wherein the total aldehyde content is 1000 ppm or less. In a preferred embodiment, the total aldehyde content is defined as the total content of aldehydes having a molecular weight of 200 g/mol or less. In another preferred embodiment, the total aldehyde content is defined as the total content of aldehydes selected from the group consisting of formaldehyde, acetaldehyde, propionaldehyde and butyraldehyde. The designation “ppm” refers to the total aldehyde content in mg per 1 kg of the composition.

As stated above, it was surprisingly found that compositions treated according to the invention and which contain compounds having at least one alkylene oxide unit in their chemical structure and a molecular weight of 200 g/mol or more, products, i. e. respective compositions, are obtained having an aldehyde content that is diminished by at least 10% of the initial value.

Preferred embodiments and preferred features, respectively, of the compositions containing compounds having at least one alkylene oxide unit in their chemical structure and a molecular weight of 200 g/mol or more, as well as the compounds themselves, are as already described above with regard to the process according to the invention.

In a preferred embodiment of the composition, the content of compounds having at least one alkylene oxide unit in their chemical structure and a molecular weight of 200 g/mol or more, is at least 10 wt %, based on the total weight of the composition, preferably at least 20 wt %, preferably at least 50 wt %, further preferably at least 80 wt %, even further preferably at least 90.0 wt %, even further preferably at least 95.0 wt %, even further preferably at least 98.0 wt %, even further preferably at least 99.0 wt %, and most preferably at least 99.5 wt %.

In another preferred embodiment of the composition, the water content of the composition is preferably 90 wt % or less, based on the total weight of the composition, further preferably 70 wt % or less, even further preferably 50 wt % or less, further preferably 40 wt % or less, further preferably 30 wt % or less, further preferably 20 wt % or less, even further preferably 10.0 wt % or less, even further preferably 5.0 wt % or less, even further preferably 2.0 wt % or less, even further preferably 1.0 wt % or less, and most preferably 0.5 wt % or less.

In another preferred embodiment of the composition, the total aldehyde content is 900 ppm or less, preferably 800 ppm or less, further preferably 500 ppm or less, even further preferably 400 ppm or less, even further preferably 300 ppm or less, even further preferably 200 ppm or less, even further preferably 150 ppm or less, even further preferably 100 ppm or less, even further preferably 50 ppm or less, and most preferably 10 ppm or less. In a particularly preferred embodiment, the residual aldehyde contents mentioned herein refer to a composition whose content of compounds having at least one aldehyde oxide unit in their chemical structure and a molecular weight of 200 mg/mol or more, is at least 10 wt % based on the total weight of the composition, particularly preferably at least 50 wt %. As already mentioned above, in a preferred embodiment, the total aldehyde content is defined as the total content of aldehydes having a molecular weight of 200 g/mol or less. In another preferred embodiment, the total aldehyde content is defined as total content of aldehydes selected from the group consisting of formaldehyde, acetaldehyde, propionaldehyde and butyraldehyde.

In another particularly preferred embodiment of the composition, the content of compounds having at least one alkylene oxide unit in their chemical structure and a molecular weight of 200 g/mol or more, is at least 10 wt %, based on the total weight of the composition, especially preferably at least 50 wt %, wherein the total aldehyde content is 1000 ppm or less, preferably 900 ppm or less, preferably 800 ppm or less, further preferably 500 ppm or less, even further preferably 400 ppm or less, even further preferably 300 ppm or less, even further preferably 200 ppm or less, even further preferably 150 ppm or less, even further preferably 100 ppm or less, even further preferably 50 ppm or less, and most preferably 10 ppm or less, wherein the total aldehyde content is defined as the total content of aldehydes having a molecular weight of 200 g/mol or less, wherein the total aldehyde content is further preferably defined as total content of aldehydes selected from the group consisting of formaldehyde, acetaldehyde, propionaldehyde and butyraldehyde. The designation “ppm” refers to the total aldehyde content in mg per 1 kg of the composition.

As mentioned above, besides aldehydes, dioxane can also be removed. Thus, in another preferred embodiment of the composition, the dioxane content is 1000 ppm or less, further preferably 900 ppm or less, preferably 800 ppm or less, further preferably 500 ppm or less, even further preferably 400 ppm or less, even further preferably 300 ppm or less, even further preferably 200 ppm or less, even further preferably 150 ppm or less, even further preferably 100 ppm or less, even further preferably 50 ppm or less, and most preferably diminished to 10 ppm or less. The dioxane is preferably selected from the group consisting of 1,2-dioxane, 1,3-dioxane and 1,4-dioxane, particularly preferably 1,4-dioxane. In a particularly preferred embodiment, the residual dioxane contents mentioned herein refer to a composition whose content of compounds having at least one alkylene oxide unit in their chemical structure and a molecular weight of 200 g/mol or more, is at least 10 wt %, based on the total weight of the composition, particularly preferably at least 50 wt %. The designation “ppm” refers to the amount of dioxane in mg per 1 kg of the composition.

Besides removing the aldehydes, the alkoxylation catalyst such as NaOH or KOH may also be removed after step a) of the process by using a cation exchanger. Therefore, in another preferred embodiment of the composition, the metal ion content is 1000 ppm or less, further preferably 900 ppm or less, preferably 800 ppm or less, further preferably 500 ppm or less, even further preferably 400 ppm or less, even further preferably 300 ppm or less, even further preferably 200 ppm or less, even further preferably 150 ppm or less, even further preferably 100 ppm or less, even further preferably 50 ppm or less, and most preferably 10 ppm or less. In a particularly preferred embodiment, the residual metal ion contents mentioned herein refer to a composition whose content of compounds having at least one alkylene oxide unit in their chemical structure and a molecular weight of 200 mg/mol or more, is at least 10 wt-%, based on the total weight of the composition, particularly preferably at least 50 wt %. In a preferred embodiment, the metal ions are selected from a group consisting of Na(I), K(I), Zn(II), Fe(II), Fe(III), Co(II), Co(III), Ni(II), Mn(II), Mn(III), Ir(III), Rh(III), Ru(II), V(IV), V(V), Co(II), Sn(II), Pb(II), Mo(IV), (Mo(VI), Al(III), V(IV), V(V), Sr(II), W(IV), W(VI), Cu(II), Cr(II) and Cr(III). The designation “ppm” refers to the amount of metal ions in mg per 1 kg of the composition.

The following Table A lists further preferred embodiments of the composition according to the invention, wherein the composition comprising at least one compound having at least one alkylene oxide unit in its chemical structure and a molecular weight of 200 g/mol or more, is defined with regard to the combination of the achieved or achievable, respectively, aldehyde and dioxane contents, or aldehyde, dioxane and metal ion contents, respectively. The numeric values are denoted in ppm and represent the stated numeric values in ppm or less. Preferably, the content of compounds having at least on alkylene oxide unit in their chemical structure and a molecular weight of 200 g/mol or more, is at least 10 wt %, based on the total weight of the composition, particularly preferably at least 50 wt %. Accordingly, the following Table A also lists preferred embodiments of the process with regard to the composition obtained after step b) having diminished contents of unwanted impurities (aldehyde and dioxane; aldehyde, dioxane and metal ions), as stated in the table.

TABLE A
Preferred embodiments of the process and of the composition
with regard to aldehyde, dioxane and metal ion impurities.

| | Total aldehyde | | |
|---|---|---|---|
| Preferred | content [X ppm or | Dioxane content | Metal ion content |
| embodiment | less] | [X ppm or less] | [X ppm or less] |
| 1 | 1000 | 1000 | |
| 2 | 900 | 900 | |
| 3 | 800 | 800 | |
| 4 | 500 | 500 | |
| 5 | 400 | 400 | |
| 6 | 300 | 300 | |
| 7 | 200 | 200 | |
| 8 | 150 | 150 | |
| 9 | 100 | 100 | |
| 10 | 50 | 50 | |
| 11 | 10 | 10 | |
| 12 | 1000 | 1000 | 1000 |
| 13 | 900 | 900 | 900 |
| 14 | 800 | 800 | 800 |
| 15 | 500 | 500 | 500 |
| 16 | 400 | 400 | 400 |
| 17 | 300 | 300 | 300 |
| 18 | 200 | 200 | 200 |
| 19 | 150 | 150 | 150 |
| 20 | 100 | 100 | 100 |
| 21 | 50 | 50 | 50 |
| 22 | 10 | 10 | 10 |

In another preferred embodiment of the composition, the compounds having at least one alkylene oxide unit in their chemical structure and a molecular weight of 200 g/mol or more, have at least one (poly)alkylene oxide group with at least 1, preferably with at least 2, preferably with at least 3, further preferably with at least 4, and even further preferably with at least 5 alkylene oxide units, even further preferably with at least 6, even further preferably with at least 8, even further preferably with at least 10, even further preferably with at least 12, even further preferably with at least 15, even further preferably with at least 50, and most preferably with at least 100 alkylene oxide units. The upper limit of the alkylene oxide units is not particularly limited. In preferred embodiments, the number of alkylene oxide units is 500 or fewer, further preferably 200 or fewer, and even further preferably 150 or fewer. Said alkylene oxide units are preferably selected from the group consisting of ethylene oxide, propylene oxide and butylene oxide, particularly preferably ethylene oxide and/or propylene oxide.

In another preferred embodiment of the composition, the compounds having at least one alkylene oxide unit in their chemical structure and a molecular weight of 200 g/mol or more, have at least one (poly)alkylene oxide group of the formula —(C₂H₄—O)ₗ—(C₃H₆—O)ₘ—(C₄H₈—O)ₙ— or —(C₂H₄—O)ₗ—(C₃H₆—O)ₘ—(C₄H₈—O)ₙ—H, wherein l, m and n independently of each other each are 0 or more, and wherein the sum of l, m and n is 1 or more, preferably, the sum of l, m and n is 2 or more, preferably, the sum of l, m and n is 3 or more, further preferably 4 or more, even further preferably 5 or more, even further preferably 6 or more, even further preferably 8 or more, even further preferably 10 or more, even further preferably 12 or more, particularly preferably 15 or more, especially preferably 50 or more, and most preferably 100 or more. The upper limit of the alkylene oxide units is not particularly limited. In further embodiments, it is preferred that the sum of l, m and n is a number from 1 to 500, further preferably, the sum of l, m and n is a number from 1 to 200, and even further preferably, the sum of l, m and n is a number from 1 to 150. In another particularly preferred embodiment, the sum of l, m and n is a number from 2 to 500, preferably, the sum of l, m and n is a number from 2 to 200, and particularly preferably, the sum of l, m and n is a number from 2 to 150.

In another preferred embodiment of the composition, the compounds having at least one aldehyde unit in their chemical structure and a molecular weight of 200 g/mol or more, have at least one (poly)alkylene oxide group of the formula-(CₓH₂ₓ—O)_{y}— or —(CₓH₂ₓ—O)_{y}—H, wherein x is 2, 3 or 4 and y is 1 or more, preferably, y is 2 or more, 3 or more, 4 or more, 5 or more, 6 or more, 8 or more, 10 or more, 12 or more, 15 or more, 50 or more, 100 or more. The upper limit of the alkylene oxide units is not particularly limited. In further embodiments, it is preferred that y is a number of 1 to 500, further preferably, y is a number of 1 to 200, and even more preferably, y is a number of 1 to 150. In another particularly preferred embodiment, y is a number of 2 to 500, preferably, y is a number of 2 to 200, and particularly preferably, y is a number of 2 to 150.

In another preferred embodiment of the composition, the molecular weight of the compounds having at least one alkylene oxide unit in their chemical structure, is 250 g/mol or more, preferably 300 g/mol or more, further preferably 400 g/mol or more, even more preferably 500 g/mol or more, especially preferably 1,000 g/mol or more, and particularly preferably 2,000 g/mol or more. The upper limit of the molecular weight of the compounds having at least one alkylene oxide unit in their chemical structure is not particularly limited. In preferred embodiments, the molecular weight of the compounds having at least one alkylene oxide unit in their chemical structure, is 100,000 g/mol or less, preferably 50,000 g/mol or less, further preferably 25,000 or less, even further preferably 20,000 g/mol or less, especially preferably 15,000 or less, and particularly preferably 12,000 g/mol or less.

In another preferred embodiment of the composition, the alkylene oxide units of the compounds having at least one alkylene oxide unit in their chemical structure and a molecular weight of 200 g/mol or more, are selected from the group consisting of ethylene oxide, propylene oxide and butylene oxide.

In another preferred embodiment of the composition, the compounds having at least one alkylene oxide unit in their chemical structure and a molecular weight of 200 g/mol or more, represent (poly)alkylene oxide adducts having at least one further residue selected from the group consisting of carboxylic acid residues, carboxylic acid ester residues, carboxamide residues, phenol residues and alcohol residues. Said alkylene oxide units are preferably selected from the group consisting of ethylene oxide, propylene oxide and butylene oxide, particularly preferably ethylene oxide and/or propylene oxide.

In another preferred embodiment of the composition, the compounds having at least one alkylene oxide unit in their chemical structure and a molecular weight of 200 g/mol or more, are selected from the group consisting of polyglycols, polyalkylene glycols, block copolymers, carboxylic acid alkoxylates, carboxylic acid polyglycol esters, alkoxylated carboxylic acid esters, alkoxylated carboxamides, alkoxylated phenols and alcohol alkoxylates. Said alkylene oxide units are preferably selected from the group consisting of ethylene oxide, propylene oxide and butylene oxide, particularly preferably ethylene oxide and/or propylene oxide.

In another preferred embodiment of the composition, the compounds having at least one alkylene oxide unit in their chemical structure and a molecular weight of 200 g/mol or more, are polyglycols of the formula H—[O—C₂H₄]ₚ—[O—C₃H₆]_{q}—[O—C₄H₈]ᵣ—OH, wherein p, q and r independently of each other each are 0 or more, and the sum of p, q and r is 5 or more, preferably, the sum of p, q and r is 6 or more, preferably 8 or more, further preferably 10 or more, even further preferably 12 or more, particularly preferably 15 or more, especially preferably 50 or more, and most preferably 100 or more. The upper limit of the alkylene oxide units is not particularly limited. In further embodiments, it is preferred that the sum of p, q and r is a number from 5 to 500, further preferably, the sum of p, q and r is a number from 5 to 200, and even further preferably, the sum of p, q and r is a number from 5 to 150. In another particularly preferred embodiment, the sum of p, q and r is a number from 10 to 500, preferably, the sum of p, q and r is a number from 10 to 200, and particularly preferably, the sum of p, q and r is a number from 10 to 150.

### DESCRIPTION OF THE FIGURES

FIG. 1 shows a schematic arrangement for a continuous process for the diminution of aldehydes with two heatable double-jacketed columns.

FIG. 2 shows a schematic arrangement for a continuous process for the diminution of aldehydes with a heatable double-jacketed column and an evaporation unit.

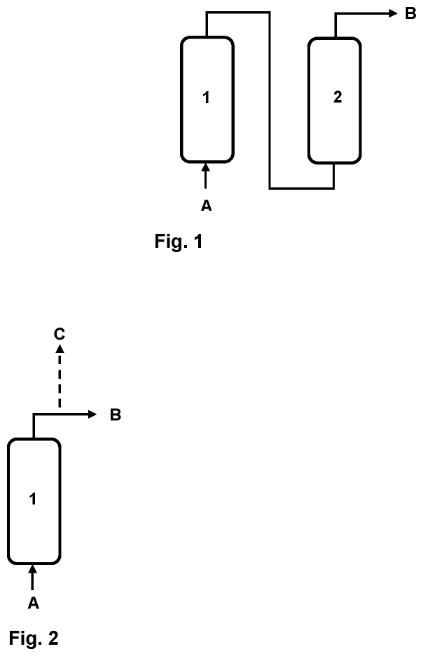

FIG. 1 shows a schematic arrangement for a continuous process for the diminution of aldehydes by using two heatable double-jacketed stainless-steel columns. The product stream (A) to be purified is conducted through the two sequentially connected columns (1, 2), the first column (1) containing an acidic SO₃H ion exchanger, the downstream column (2) containing a basic NH₂ ion exchanger. The two columns (1, 2) may, for example, be operated at a temperature of 100° C. The obtained purified product stream (B) contains diminished aldehyde contents.

FIG. 2 shows a schematic arrangement for a continuous process in which the product stream (A) to be purified is conducted through a heatable double-jacketed column (1) containing an acidic SO₃H ion exchanger. At the outlet of column (1), an evaporation unit is installed that allows for removing the released aldehydes from the outgoing product stream via the gas phase by way of a vacuum (C). The column (1) may, for example, be operated at a temperature of 100° C. The obtained purified product stream (B) contains diminished aldehyde contents.

The following examples illustrate the features and advantages of the invention by way of different embodiments.

### EXAMPLES

In the scope of the invention, it was investigated how to diminish the aldehyde content in polyglycol compounds and in various alkoxylates that were prepared for experimental purposes at high alkoxylation temperatures. For the experiments, as a cation exchanger a sulfonic-acid-functional ion exchanger “Lewatit SP 112 H” by Lanxess was used which is referred to as “SO₃H ion exchanger” in the examples. As a basic ion exchanger, hereinafter called “NH₂ ion exchanger”, the product “Lewatit VP OC 1065” by Lanxess was used. The aldehydes contained in the chemical compositions were separated from other substances via HPLC after derivatization with a 2,4-dinitrophenyl hydrazine solution and measured and quantified by way of UV detection (according to B. Reindl, H.-J. Stan, Agric. Food Chem., 30 (1982) 849-854, and J. R. Dahlgran, M. N. Jameson, J. Assoc. Off. Anal. Chem., 71/3 (1988) 560-563). In the following examples, the designation “ppm” refers to the amount of the measured substance or the measured ions, respectively, in mg per 1 kg of the composition.

### Example 1: Treatment of Alkoxylated Rapeseed Oil (18 EO/6 PO) with SO₃H and NH₂ Ion Exchangers

To study the influence of the ion exchanger on the diminution of aldehydes, 100 g of alkoxylated rapeseed oil having 18 EO (corresponding to 18 ethylene oxide units) and 6 PO (corresponding to 6 propylene oxide units) were stirred in a 150 ml beaker at 100° C. during 30 min. The experiment was performed without and with 10 wt % of the acidic SO₃H and the basic NH₂ ion exchanger, respectively. In case the ion exchanger was present, it was removed after the treatment and the remaining acetaldehyde and propionaldehyde content was determined in the remaining product. The results are summarized in Table 1. Before the treatment, the alkoxylated rapeseed oil having 18 EO and 6 PO had an acetaldehyde content of 6840 ppm and a propionaldehyde content of 805 ppm. From this experiment, it is apparent that a significant aldehyde diminution is made possible by the acidic SO₃H ion exchanger. The basic NH₂ ion exchanger alone did not yield any substantial aldehyde diminution.

The number-average molecular weight (Mn) of the employed alkoxylated rapeseed oil was calculated as follows: 299.3 g/mol (rapeseed oil)+18×44.05 g/mol (ethylene oxide)+6×58.08 g/mol (propylene oxide)=1,440.6 g/mol. The molecular weight of the rapeseed oil was derived from the saponification value of 187.5 mg KOH/g according to DIN EN ISO 3681, DGF C-V3.

TABLE 1
Influence of the ion exchanger on the aldehyde content in an
alkoxylated rapeseed oil having 18 EO and 6 PO (initial value
for acetaldehyde: 6840 ppm, propionaldehyde: 805 ppm).

| | Dwell time | Temperature | Acetaldehyde | Propionaldehyde |
|---|---|---|---|---|
| | [min] | [° C.] | [ppm] | [ppm] |
| Initial value | | | 6840 | 805 |
| Without ion | 30 | 100 | 6700 | 829 |
| exchanger | | | | |
| 10% NH₂ ion | 30 | 100 | 6430 | 858 |
| exchanger | | | | |
| 10% SO₃H ion | 30 | 100 | 864 | 152 |
| exchanger | | | | |

### Example 2a: Treatment of Alkoxylated Rapeseed Oil (18 EO/6 PO) with SO₃H and NH₂ Ion Exchangers

To study the influence of a combination of an acidic and a subsequently used basic ion exchanger on the diminution of aldehydes, 100 g alkoxylated rapeseed oil having 18 EO and 6 PO were stirred in a 150 ml beaker at 60° C., 80° C. and 100° C., respectively, for 15 and 30 min, respectively, with 5 and 10 wt %, respectively, of the acidic SO₃H ion exchanger (process step A). After the treatment, the ion exchanger was separated from the product and a sample was taken to determine the aldehyde content. In the second process step B, 5 and 10 wt %, respectively, of the basic NH₂ ion exchanger were added to the product and stirred at different temperatures for 15 and 30 min, respectively. Subsequently, the ion exchanger was removed and the aldehyde content was determined. Before the treatment, the alkoxylated rapeseed oil having 18 EO and 6 PO had an acetaldehyde content of 7050 ppm and a propionaldehyde content of 990 ppm. The results are summarized in Table 2a. From this series of experiments, it is apparent that by a combined treatment of first using an acidic ion exchanger and then a basic NH₂ ion exchanger, a significant diminution of aldehydes is possible, a higher percentage of ion exchanger and a higher temperature of 100° C. facilitating the diminution.

The number-average molecular weight (MN) of the employed alkoxylated rapeseed oil was calculated as follows: 299.3 g/mol (rapeseed oil)+18×44.05 g/mol (ethylene oxide)+6×58.08 g/mol (propylene oxide)=1,440.6 g/mol. The molecular weight of the rapeseed oil was derived from the saponification value of 187.5 mg KOH/g according to DIN EN ISO 3681, DGF C-V3.

TABLE 2a
Influence of a combination of ion exchangers on the aldehyde content
in an alkoxylated rapeseed oil + 18 EO + 6 PO.

| | | | | Acetaldehyde | Propionaldehyde |
|---|---|---|---|---|---|
| | | | | [ppm] | [ppm] |
| Initial Value | | | | 7050 | 990 |
| | Percentage | | | | |
| | SO₃H ion | | | | |
| Process | exchanger | Dwell time | Temperature | | |
| stage A | [%] | [min] | [° C.] | | |
| A1) | 10 | 30 | 100 | 458 | 248 |
| A2) | 5 | 15 | 80 | 5280 | 742 |
| A3) | 5 | 15 | 60 | 6000 | 877 |
| | Percentage | | | | |
| | NH₂ ion | | | | |
| Process | exchanger | Dwell time | Temperature | Acetaldehyde | Propionaldehyde |
| stage B | [%] | [min] | [° C.] | [ppm] | [ppm] |
| A1 → B1) | 10 | 30 | 100 | Not | Not |
| | | | | detectable | detectable |
| A2 → B2) | 5 | 15 | 80 | 4020 | 607 |
| A3 → B3) | 5 | 15 | 60 | 4980 | 678 |

### Example 2b: Treatment of Alkoxylated Sorbitan Monolaurate (20 EO) with SO₃H and NH₂ Ion Exchangers

The experimental procedure corresponds to Example 2a), wherein, instead of the alkoxylated rapeseed oil having 18 EO and 6 PO, a sorbitan monolaurate having 20 EO was tested. Before the treatment, the sorbitan monolaurate having 20 EO had an acetaldehyde content of 1070 ppm. The results are summarized in Table 2a. From this series of experiments, it is apparent that by a combined treatment with an acidic ion exchanger and subsequently a basic ion exchanger, a significant aldehyde content diminution is made possible.

The number-average molecular weight (MN) of the employed alkoxylated sorbitan monolaurate was calculated as follows: 541.5 g/mol (sorbitan monolaurate)+20×44.05 g/mol (ethylene oxide)=1,422.5 g/mol.

TABLE 2b
Influence of a combination of ion exchangers on the aldehyde
content in an ethoxylated sorbitan monolaurate + 20 EO.

| | | | | Acetaldehyde |
|---|---|---|---|---|
| | | | | [ppm] |
| Initial Value | | | | 7050 |
| | Percentage | | | |
| | SO₃H ion | | | |
| Process | exchanger | Dwell time | Temperature | |
| stage A | [%] | [min] | [° C.] | |
| A1) | 10 | 30 | 100 | 159 |
| A2) | 10 | 15 | 100 | 408 |
| A3) | 5 | 30 | 100 | 230 |
| | Percentage | | | |
| | NH₂ ion | | | |
| Process | exchanger | Dwell time | Temperature | Acetaldehyde |
| stage B | [%] | [min] | [° C.] | [ppm] |
| A1 → B1) | 10 | 30 | 100 | 36 |
| A2 → B2) | 10 | 15 | 100 | 252 |
| A3 → B3) | 5 | 30 | 100 | 38 |

### Example 2c: Treatment of Alkoxylated Isotridecyl Alcohol (7 EO) with SO₃H and NH₂ Ion Exchangers

The experimental procedure corresponds to Example 2a), wherein, instead of the alkoxylated rapeseed oil having 18 EO and 6 PO, an isotridecyl alcohol having 7 PO was tested. Before treatment, the isotridecyl alcohol having 7 EO had an acetaldehyde content of 17 ppm. The results are summarized in Table 2c. From this series of experiments, it is apparent that by a combined treatment with an acidic ion exchanger and subsequently a basic ion exchanger a significant aldehyde diminution is made possible.

The number-average molecular weight (MN) of the employed ethoxylated isotridecyl alcohol was calculated as follows: 196.9 g/mol (isotridecyl alcohol)+7×44.05 g/mol (ethylene oxide)=505.2 g/mol. The molecular weight of the isotridecyl alcohol was derived from the hydroxyl value of 285 mg KOH/g according to Ph. Eur. 2.5.3.

TABLE 2c
Influence of a combination of ion exchangers on the aldehyde
content in an ethoxylated isotridecyl alcohol + 7 EO.

| | | | | Acetaldehyde |
|---|---|---|---|---|
| | | | | [ppm] |
| Initial Value | | | | 17 |
| | Percentage | | | |
| | SO₃H ion | | | |
| Process | exchanger | Dwell time | Temperature | |
| stage A | [%] | [min] | [° C.] | |
| A1) | 10 | 30 | 100 | 11 |
| A2) | 5 | 30 | 100 | 10 |
| | Percentage | | | |
| | NH₂ ion | | | |
| Process | exchanger | Dwell time | Temperature | Acetaldehyde |
| stage B | [%] | [min] | [° C.] | [ppm] |
| A1 → B1) | 10 | 30 | 100 | 5 |
| A2 → B2) | 5 | 30 | 100 | 5 |

### Reference Example 3a: Treatment of Ethoxylated Cocoyl Acid (9 EO) by Way of Steam Distillation

In a steam distillation apparatus consisting of a 500 ml flask with a distillation attachment, a steam inlet pipe and a stirring unit, 350 g of ethoxylated cocoyl acid having 9 EO were weighed in. Under stirring, the product was heated to 105° C., and when 105° C. had been reached, saturated steam was introduced during 240 min, the distillate being collected by way of a receiver. When the temperature of 105° C. had been reached, a sample was taken (corresponding to a dwell time of 0 min) and also after a steam treatment of 60 and 240 min at 105° C., respectively. The samples were analyzed with regard to their aldehyde and 1,4-dioxane content. The results are summarized in Table 3a. Before treatment, the cocoyl acid ethoxylate had a formaldehyde content of 58 ppm, an acetaldehyde content of 2740 ppm and a 1,4-dioxane content of 3200 ppm.

The number-average molecular weight (MN) of the employed cocoyl acid ethoxylate was calculated as follows: 209.0 g/mol (cocoyl acid)+9×44.05 g/mol (ethylene oxide)=605.4 g/mol. The molecular weight of the cocoyl acid was derived from the acid value of 268.5 mg KOH/g according to DIN EN ISO 3682.

TABLE 3a
(Reference Example): Aldehyde and 1,4-dioxane content of
an ethoxylated cocoyl acid having 9 EO that was treated
at 105° C. during 240 min using steam distillation.

| | | | | | 1,4- |
|---|---|---|---|---|---|
| | | | Formaldehyde | Acetaldehyde | Dioxane |
| | | | [ppm] | [ppm] | [ppm] |
| Initial value | | | 58 | 2740 | 3200 |
| Percentage | | | | | |
| SO₃H ion | | | | | |
| exchanger | Dwell time | Temperature | | | |
| [%] | [min] | [° C.] | | | |
| None | 0 | 105 | <53 | 2740 | 3200 |
| None | 60 | 105 | 24 | 2390 | 350 |
| None | 240 | 105 | <17 | 2240 | 1 |

### Example 3b: Treatment of Ethoxylated Cocoyl Acid (9 EO) by Using an SO₃H Ion Exchanger and Steam Distillation

The experimental procedure corresponds to Example 3a, wherein initially 2 wt % of the acidic SO₃H ion exchanger were added to the ethoxylated cocoyl acid having 9 EO. The results are summarized in Table 3b. A comparison of Table 3b with Table 3a shows that the presence of an acidic SO₃H ion exchanger had a significant influence on the aldehyde content diminution. The 1,4-dioxane content could also be diminished.

TABLE 3b
Aldehyde and 1,4-dioxane content of an ethoxylated cocoyl acid
having 9 EO treated by way of a steam distillation at 105° C.
in the presence of an SO₃H ion exchanger during 240 min.

| | | | | | 1,4- |
|---|---|---|---|---|---|
| | | | Formaldehyde | Acetaldehyde | Dioxane |
| | | | [ppm] | [ppm] | [ppm] |
| Initial value | | | 58 | 2740 | 3200 |
| Percentage | | | | | |
| SO₃H ion | | | | | |
| exchanger | Dwell time | Temperature | | | |
| [%] | [min] | [° C.] | | | |
| 2 | 0 | 105 | <17 | 2550 | 3100 |
| 2 | 60 | 105 | <17 | Not | 56 |
| | | | | detectable | |
| 2 | 240 | 105 | <17 | Not | 2 |
| | | | | detectable | |

### Example 3c: Treatment of Ethoxylated Cocoyl Acid (9 EO) Using Methanesulfonic Acid and Steam Distillation

The experimental procedure corresponds to Example 3a, wherein initially 0.5 wt % of methanesulfonic acid were added to the cocoyl acid having 9 EO. The results are summarized in Table 3c. A comparison of Table 3c with Table 3a shows that the presence of methanesulfonic acid had a significant influence on the aldehyde content diminution. The 1,4-dioxane content could also be diminished.

TABLE 3c
Aldehyde and 1,4-dioxane content of an ethoxylated cocoyl acid
having 9 EO that was treated using steam distillation at 105°
C. in the presence of methanesulfonic acid during 240 min.

| | | | | | 1,4- |
|---|---|---|---|---|---|
| | | | Formaldehyde | Acetaldehyde | Dioxane |
| | | | [ppm] | [ppm] | [ppm] |
| Initial value | | | 58 | 2740 | 3200 |
| Percentage | | | | | |
| methane- | | | | | |
| sulfonic | Dwell time | Temperature | | | |
| acid [%] | [min] | [° C.] | | | |
| 0.5 | 0 | 105 | <17 | 1920 | 2900 |
| 0.5 | 60 | 105 | 43 | 24 | 1100 |
| 0.5 | 240 | 105 | <17 | <22 | 15 |

### Reference Example 4a: Treatment of Ethoxylated Cocoyl Acid (9 EO) Using Distillation

In a distillation apparatus consisting of a 500 ml flask with a distillation attachment and a stirring unit, 350 g of ethoxylated cocoyl acid having 9 EO were weighed in and 35 g of water were added. The employed cocoyl acid having 9 EO was prepared by ethoxylation of cocoyl acid with ethylene oxide with the aid of caustic potash as a catalyst. The mixture of cocoyl acid having 9 EO and water was heated to 80° C. under stirring, and when a temperature of 80° C. had been reached, a sample was taken (corresponding to a dwell time of 0 min). Then, a vacuum (final pressure: 200 mbar) was applied and the water, including byproducts, were distilled off. Samples were taken after 60 and 240 min, respectively, breaking the vacuum. The samples were analyzed with regard to their contents of aldehyde, 1,4-dioxane and potassium. The results are summarized in Table 4a. Before treatment, the cocoyl acid ethoxylate had a formaldehyde content of 58 ppm, an acetaldehyde content of 2740 ppm, a 1,4-dioxane content of 3200 ppm and a potassium content of 1100 ppm.

The number-average molecular weight (MN) of the employed ethoxylated cocoyl acid was calculated as follows: 209.0 g/mol (cocoyl acid)+9×44.05 g/mol (ethylene oxide)=605.4 g/mol. The molecular weight of the cocoyl acid was derived from the acid value of 268.5 mg KOH/g according to DIN EN ISO 3682.

TABLE 4a
(Reference Example): Aldehyde-, 1,4-dioxan- and potassium content
of an ethoxylated cocoyl acid having 9 EO treated by distillation
at 80° C. under vacuum with 10 wt % water during 240 min.

| | | | Formal- | Acetal- | 1,4- | Potas- |
|---|---|---|---|---|---|---|
| | | | dehyde | dehyde | Dioxane | sium |
| | | | [ppm] | [ppm] | [ppm] | [ppm] |
| Initial | | | 58 | 2740 | 3200 | 1100 |
| value | | | | | | |
| Percentage | | | | | | |
| SO₃H ion | Dwell | Temper- | | | | |
| exchanger | time | ature | | | | |
| [%] | [min] | [° C.] | | | | |
| None | 0 | 80 | 69 | 2460 | 2600 | 1100 |
| | | (200 mbar) | | | | |
| None | 60 | 80 | 67 | 2610 | 700 | |
| | | (200 mbar) | | | | |
| None | 240 | 80 | 69 | 2680 | 200 | 1100 |
| | | (200 mbar) | | | | |

### Example 4b: Treatment of Ethoxylated Cocoyl Acid (9 EO) Using an SO₃H Ion Exchanger and Distillation

The experimental procedure corresponds to Example 4a, wherein initially 2 wt % of the acidic SO₃H ion exchanger were added with water to the ethoxylated cocoyl acid having 9 EO. The results are summarized in Table 4b. A comparison of Table 4b with Table 4a shows that the presence of an acidic SO₃H ion exchanger has a significant influence on the aldehyde content diminution. The 1,4-dioxane content could also be diminished. Furthermore, by using a cation ion exchanger, the potassium content could be diminished significantly, the potassium ions originating from the use of caustic potash as a catalyst in the ethoxylation reaction. Thus, by applying the process according to the invention, products can be prepared that have a diminished content of aldehydes, 1,4-dioxane and cations, with respect to the initial value.

TABLE 4b
Aldehyde, 1,4-dioxane and potassium content of an ethoxylated
cocoyl acid having 9 EO that was treated by distillation at
80° C. under vacuum in the presence of an SO₃H ion
exchanger and 10 wt % water during 240 min.

| | | | Formal- | Acetal- | 1,4- | Potas- |
|---|---|---|---|---|---|---|
| | | | dehyde | dehyde | Dioxane | sium |
| | | | [ppm] | [ppm] | [ppm] | [ppm] |
| Initial | | | 58 | 2740 | 3200 | 1100 |
| value | | | | | | |
| Percentage | | | | | | |
| SO₃H ion | Dwell | Temper- | | | | |
| exchanger | time | ature | | | | |
| [%] | [min] | [° C.] | | | | |
| 2 | 0 | 80 | <59 | 2250 | 2900 | 1100 |
| | | (200 mbar) | | | | |
| 2 | 60 | 80 | <59 | 605 | 406 | |
| | | (200 mbar) | | | | |
| 2 | 240 | 80 | <15 | 30 | 1 | <35 |
| | | (200 mbar) | | | | |

### Example 5: Purification of Alkoxylated Rapeseed Oil (18 EO/6 PO) Via Columns Equipped with an SO₃H Ion Exchanger and an NH₂ Ion Exchanger, Respectively

For the experiment, a continuous process for diminishing aldehydes was used, wherein two heatable sequentially connected double-jacketed stainless-steel columns (as shown in FIG. 1) having a volume of about 250 cm³ as well as a feed pump having a flow rate of about 2 g/min were employed. The first column was filled with about 150 g of the acidic SO₃H ion exchanger and the downstream column was filled with about 150 g of a basic NH₂ ion exchanger. The two columns were operated at a temperature of 100° C. The liquid product stream consisted of an alkoxylated rapeseed oil having 18 EO and 6 PO, with initial values of 6840 ppm acetaldehyde and 805 ppm propionaldehyde. After a lead time of 2 h, every hour a sample of about 20 g was taken from the outgoing product stream which was tested with regard to its aldehyde content. The results are summarized in Table 5. The results from Table 5 show that the aldehyde contents can be diminished by more than 90% under the aforementioned conditions.

The number-average molecular weight (MN) of the employed alkoxylated rapeseed oil was calculated as follows: 299.3 g/mol (rapeseed oil)+18×44.05 g/mol (ethylene oxide)+6×58.08 g/mol (propylene oxide)=1,440.6 g/mol. The molecular weight of the rapeseed oil was derived from the saponification value of 187.5 mg KOH/g according to DIN EN ISO 3681, DGF C-V3.

TABLE 5
Temporal development of the aldehyde content in an alkoxylated
rapeseed oil having 18 EO and 6 PO treated according
to a continuous process according to FIG. 1.

| | | Acetaldehyde | Propionaldehyde |
|---|---|---|---|
| | | [ppm] | [ppm} |
| Initial value | | 6840 | 805 |
| Operating | Temperature | | |
| time [h] | [° C.] | | |
| 2 | 100 | 458 | Not detectable |
| 3 | 100 | 470 | Not detectable |
| 4 | 100 | 495 | Not detectable |
| 5 | 100 | 452 | Not detectable |
| 6 | 100 | 408 | Not detectable |

### Example 6: Purification of Polyethylene Glycol 400 Via Columns Equipped with an SO₃H Ion Exchanger and an NH₂ Ion Exchanger, Respectively

The experimental procedure was performed in analogy to Example 5, the product stream consisting of polyethylene glycol 400 having an initial formaldehyde content value of 68 ppm. The results are summarized in Table 6. The results from Table 6 show that the formaldehyde content can be diminished by more than 90% under the aforementioned conditions.

The number-average molecular weight (MN) of the employed polyethylene glycol 400 of 399.4 g/mol was derived from the hydroxyl value of 281 mg KOH/g according to Ph. Eur. 2.5.3.

TABLE 6
Temporal development of the formaldehyde content
in a polyethylene glycol 400 treated according
to a continuous process according to FIG. 1.

| | | Formaldehyde [ppm] |
|---|---|---|
| Initial value | | 68 |
| Operating time [h] | Temperature [° C.] | |
| 2 | 60 | <3 |
| 3 | 60 | Not detectable |
| 4 | 60 | Not detectable |
| 5 | 60 | Not detectable |
| 6 | 60 | 4 |

### Example 7: Purification of Alkoxylated Rapeseed Oil (18 EO/6 PO) Via a Column Equipped with an SO₃H Ion Exchanger and an Evaporation Unit Attached Downstream

For the experiment, a continuous process for diminishing aldehydes was applied, employing a heatable double-jacketed stainless-steel column (as shown in FIG. 2) having a volume of about 250 cm³, as well as a feed pump having a flow rate of about 2 g/min. At the column's outlet, an evaporation unit was installed which allowed for removing the released aldehydes via the gas phase by way of a vacuum (about 200 mbar). The column was filled with about 150 g of the acidic SO₃H ion exchanger. The column was operated at a temperature of 100° C. The liquid product stream consisted of an alkoxylated rapeseed oil having 18 EO and 6 PO, the initial values of acetaldehyde being 6440 ppm and propionaldehyde being 818 ppm. After a lead time of 2 h, every hour a sample of about 20 g was taken from the outgoing product stream which was tested with regard to its aldehyde content. The results are summarized in Table 7.

The results in Table 7 show that the aldehyde content can be diminished by more than 60% under the aforementioned conditions.

The number-average molecular weight (MN) of the employed alkoxylated rapeseed oil was calculated as follows: 299.3 g/mol (rapeseed oil)+18×44.05 g/mol (ethylene oxide)+6×58.08 g/mol (propylene oxide)=1,440.6 g/mol. The molecular weight of the rapeseed oil was derived from the saponification value of 187.5 mg KOH/g according to DIN EN ISO 3681, DGF C-V3.

TABLE 7
Temporal development of the aldehyde content in an alkoxylated
rapeseed oil having 18 EO and 6 PO, treated according
to a continuous process according to FIG. 2.

| | | Acetaldehyde | Propionaldehyde |
|---|---|---|---|
| | | [ppm] | [ppm} |
| Initial value | | 6440 | 818 |
| Operating | Temperature | | |
| time [h] | [° C.] | | |
| 2 | 100 | 982 | 180 |
| 3 | 100 | 1370 | 222 |
| 4 | 100 | 1360 | 271 |
| 5 | 100 | 1490 | 174 |
| 6 | 100 | 1780 | 184 |

1. A process for at least partially removing aldehydes from a composition, the process comprising the following steps:
a) contacting the composition with at least one cation exchanger at a temperature of 20.0° C. to 250.0° C.;
b) at least partially separating the aldehydes from the composition and, optionally, treatment of the separated aldehydes, selected from condensation, absorption, adsorption, chemical bonding, chemical reaction, oxidation and pyrolysis;
whereby a composition having a diminished content of aldehydes is obtained;
wherein the composition to be treated in step a) contains at least one compound having at least one alkylene oxide unit in its chemical structure and a molecular weight of 200 g/mol or more, and
3.5 wt % or less of alcohols having 1 to 6 carbon atoms, based on the total weight of the composition.

2. The process according to claim 1, characterized in that step a) is carried out at a temperature of 40° C. to 140° C.

3. The process according to claim 1, characterized in that the cation exchanger used in step a) is an acidic cation exchanger containing acid groups selected from —SO₃H, —COOH and —OP(OH)₃.

4. (canceled)

5. The process according to claim 1, characterized in that step b) is carried out by a thermal separation process, selected from the group consisting of distillation process, rectification process, stripping process and flash evaporation process.

6. The process according to claim 1, characterized in that step b) is carried out by contacting an ion exchanger having amino or bisulfite functional groups, and/or wherein step b) is carried out at a temperature of 20° C. to 250° C.

7. The process according to claim 1, characterized in that the content of compounds having at least one alkylene oxide unit in their chemical structure and a molecular weight of 200 g/mol or more, is at least 10 wt % in the employed composition, based on the total weight of the composition.

8. The process according to claim 1, characterized in that after step b) a composition is obtained the total aldehyde content of which is diminished by at least 10% of the initial value.

9. The process according to claim 1, characterized in that after step b) a composition is obtained the total aldehyde content of which is diminished to 1000 ppm or less.

10. The process according to claim 1, characterized in that step b) is carried out by a thermal separation process selected from the group consisting of distillation process, rectification process, stripping process and flash evaporation process, wherein, after step b), a composition is obtained the dioxane content of which is diminished to 1000 ppm or less.

11. The process according to claim 1, characterized in that the compounds having at least one alkylene oxide unit in their chemical structure and a molecular weight of 200 g/mol or more, have at least one (poly)alkylene oxide group, with at least 1 alkylene oxide units.

12. The process according to claim 1, characterized in that the compounds having at least one alkylene oxide unit in their chemical structure and a molecular weight of 200 g/mol or more, represent (poly)alkylene oxide adducts having at least one further residue selected from the group consisting of carboxylic acid residues, carboxylic acid ester residues, carboxamide residues, phenol residues and alcohol residues.

13. The process according to claim 1, characterized in that the compounds having at least one alkylene oxide unit in their chemical structure and a molecular weight of 200 g/mol or more, are selected from the group consisting of polyglycols, polyalkylene glycols, block copolymers, carboxylic acid alkoxylates, carboxylic acid polyglycol esters, alkoxylated carboxylic acid esters, alkoxylated carboxamides, alkoxylated phenols and alcohol alkoxylates.

14. A composition containing at least one compound having at least one alkylene oxide unit in its chemical structure and a molecular weight of 200 g/mol or more, characterized in that the total aldehyde content is 1000 ppm or less and the dioxane content is 1000 ppm or less.

15. (canceled)

16. The composition according to claim 14, characterized in that the metal ion content is 1000 ppm or less.

17. The process according to claim 11, characterized in that the compounds having at least one alkylene oxide unit in their chemical structure and a molecular weight of 200 g/mol or more, have at least one (poly)alkylene oxide group, with at least 2 alkylene oxide units.

18. The process according to claim 11, characterized in that the compounds having at least one alkylene oxide unit in their chemical structure and a molecular weight of 200 g/mol or more, have at least one (poly)alkylene oxide group, with at least 3 alkylene oxide units.

19. The process according to claim 11, characterized in that the compounds having at least one alkylene oxide unit in their chemical structure and a molecular weight of 200 g/mol or more, have at least one (poly)alkylene oxide group, with at least 4 alkylene oxide units.

20. The process according to claim 11, characterized in that the compounds having at least one alkylene oxide unit in their chemical structure and a molecular weight of 200 g/mol or more, have at least one (poly)alkylene oxide group, with at least 5 alkylene oxide units.
